(19) United States
(12) Patent Application Publication
(10) Pub. No.: US 2017/0056553 A1
(43) Pub. Date: Mar. 2, 2017

(54) COMPOSITION INCLUDING BIOACTIVE BONE GRAFTING MATERIALS AND A METALLIC SURFACE COATING, METHOD OF MAKING AND USING THE COMPOSITION

(71) Applicant: NovaBone Products, LLC, Alachua, FL (US)

(72) Inventors: Gregory J. Pomrink, Newberry, FL (US); Zehra Tosun, Gainesville, FL (US); R. Layne Howell, Gainesville, FL (US)

(73) Assignee: NovaBone Products, LLC, Alachua, FL (US)

(21) Appl. No.: 15/251,957

(22) Filed: Aug. 30, 2016

Related U.S. Application Data
(60) Provisional application No. 62/212,845, filed on Sep. 1, 2015.

(51) Int. Cl. A61L 27/30; A61L 27/18

(52) U.S. Cl. CPC A61L 27/306; A61L 27/18; A61L 2420/00

(57) ABSTRACT

Compositions including bioactive glass ceramic material surface-coated with a metallic material and methods of making and using the metallic material-coated compositions.

### CROSS-REFERENCE TO RELATED APPLICATIONS

This application claims the benefit of U.S. Provisional Patent Application No. 62/212,845, filed Sep. 1, 2015, the entire contents of which are hereby incorporated herein by reference.

### BACKGROUND OF THE INVENTION

Bone injuries do not often occur in isolation. Often there is significant wounding and trauma to the soft tissue surrounding the injured bone. A bone fracture is often associated with significant inflammation and bruising of the surrounding tissue. It may be the case that delayed wound healing may serve to inhibit repair of the fractured bone, such as by promoting excessive and long-lasting inflammation. Thus, there is a need to combine treatments and agents promoting bone repair, such as bioactive glass, with a therapeutic agent that has anti-inflammatory effect and promotes soft tissue wound healing.

Bioactive glass was originally developed in 1969 by L. Hench. Additionally bioactive glasses were developed as bone replacement materials, with studies showing that bioactive glass can induce or aid in osteogenesis. Hench et al, J. Biomed. Mater. Res. 5:117-141 (1971). Bioactive glass can form strong and stable bonds with bone. Piotrowski et al., J. Biomed. Mater. Res. 9:47-61(1975). Further, bioactive glass is not considered toxic to bone or soft tissue from studies of in vitro and in vivo models. Wilson et al., J. Biomed. Mater. Res. 805-817 (1981). Exemplary bioactive glasses known in the art include 45S5, 45S5B1, 58S, SP53 and S70C30. The original bioactive glass, 45S5, is melt-derived. Sol-gel derived glasses have nanopores that allow for increased surface area and bioactivity. However, bioactive glass may not be sufficient for ensuring that both damaged bone and the associated wounded soft tissue are both repaired. Bioactive glass may also promote coagulation of blood, which would impair the healing of a soft tissue wound.

Metallic and gold compounds have been used for medicinal purposes historically and are still in use. For example, metal has been used extensively in the manufacturing of orthopedic implants in a multitude of different forms. Multiple different materials throughout history have been tested as replacements for bone. Materials as diverse as ivory, wood, rubber, acrylic, and Bakelite have been used in the manufacture of prosthetic implants. The extensive use in modern times of metallic alloys is related to the availability and success at the beginning of the 20th century of several different alloys made of the noble metals. Implants made from iron, cobalt, chromium, titanium, and tantalum are commonly used. Clinical studies have demonstrated that alloys made from these metals can be used safely and effectively in the manufacturing of orthopedic implants that are left in vivo for extended periods. The mechanical, biologic, and physical properties of these materials play significant roles in the longevity of these implants.

Gold (usually as the metal) is perhaps the most anciently administered medicine. In medieval times, gold was often seen as beneficial for the health, in the belief that something so rare and beautiful could not be anything but healthy. In the 19th century gold had a reputation as a “nervine,” a therapy for nervous system disorders. One of its modern applications is in the treatment of rheumatoid arthritis (aurotherapy). Despite their long history, the basis for their therapeutic properties has never been established definitively. Vercruysse et al. observed that cartilage tissue and chondroitin sulfate (CS) can generate nanoparticles of Au or silver (Ag) when incubated with ionic solutions of Au or Ag. These observations prompted Vercruysse et al. hypothesize that during aurotherapy, at the site of an arthritic joint, the ionic Au could be coordinated to the CS molecules ubiquitously present in the cartilage tissue of the joints and be reduced into metallic (nano)particles of Au(0) (Vercruysse et al., “Potential anti-inflammatory properties of biologically-synthesized nanoparticles of gold or silver,” NSTI Nanotech, The Nanotechnology Conference and Trade Show, Boston 1-5, 2008, Abstract). To test this hypothesis Vercruysse et al. generated nanoparticles of Au or Ag using cartilage tissue or CS and tested these for their potential anti-inflammatory properties in an embryonic zebrafish model for identifying nanomaterials that possess anti-inflammatory properties. Preliminary results from Vercruysse et al. indicated little toxicity from exposure to the Au or Ag nanoparticles solutions at concentrations up to 100 mM and the magnitude of the inflammatory response was significantly decreased in the presence of biologically-synthesized Au or Ag nanoparticles.

Thunus et al. demonstrated anti-inflammatory properties of copper, gold and silver, individually and as mixtures in adjuvant arthritis in the rat animal model (Thunus et al., “Anti-inflammatory properties of copper, gold and silver, individually and as mixtures,” Analyst., 120(3):967-73 (1995 March)).

Wang et al. (Wang et al., Coord. Chem. Rev., 253: 1607 (2009) has studied incorporation of gold-containing nanoparticles (AuNPs) into bioactive glass ceramics and demonstrated improvement of functionalization property of the ceramic with polymers.

Jayalekshimi and Sharma discussed the development of a biodegradable polymer encapsulated-nanogold incorporated-bioactive glass composite and suggested that it could serve as a promising material for targeted drug delivery applications (Jayalekshimi and Sharma, Colloids and Surfaces B: Biointerfaces, 126:280-287 (2015)). The study suggested that the composite can be used for incorporating in the voids formed by the removal of bone tumors for controlled drug release to suppress tumors as well as enhance further bone growth.

Lusvardi et al. discussed synthesis of bioactive glasses containing AuNPs via the sol-gel route using gold precursors.

Nonetheless, there is still a need for improved treatments and agents for promoting bone repair and soft tissue wound healing.

### SUMMARY OF THE INVENTION

One embodiment provides for a bone grafting composition comprising a bioactive glass ceramic material at least partially surface-coated with a metallic material having an atomic mass greater than 45 and less than 205. The metallic material may be selected, for example, from gold, silver, platinum, copper, palladium, iridium, strontium, cerium, or isotopes, or alloys thereof. The bioactive glass ceramic material may be in a form of a particle (including spheres), a sheet, a fiber, a mesh, or any combination of these forms. The metallic material may be physically (van der Waal forces, or hydrogen-bonding) or chemically (covalent bonds) bound to the bioactive glass ceramic material. The bioactive glass ceramic material may comprise at least one of silica, boron, and calcium phosphate. The weight ratio of the metallic material may be about 0.0001%-20% relative to the weight of the synthetic bone grafting composition. Alternatively, the weight ratio of the metallic material may be less than about 20%. The composition can comprise 0-90% silica, 0-90% boric acid, or a combination thereof. The bioactive glass ceramic material can comprise bioactive glass. The bioactive glass may be in a form of particles ranging in size from about 0.01 nm to about 5 mm. The bioactive glass can comprise SiO₂, or P₂O₅, or B₂O₃, or SiO₂ and P₂O₅, or SiO₂ and B₂O₃, or K₂O, or MgO. The bioactive glass can comprise 40-60% SiO₂, 10-20% CaO, 0-4% P₂O₅, and 19-30% NaO. The bioactive glass may further comprise a carrier selected from the group consisting of hydroxyapatite and tricalcium phosphate. The bioactive glass ceramic material may be pretreated in a solution comprising one or more of blood, bone marrow aspirate, bone-morphogenetic proteins, platelet-rich plasma, and osteogenic proteins. The composition may be in a form of a gel, putty, or a solid (e.g., sphere, wedge, block, plug, and particle) at a room temperature. The composition is osteoinductive. The composition conducts an electrical current. The composition promotes more rapid wound healing as compared to a composition having uncoated bioactive glass ceramic material. The metallic material coating mount ranges from about 1 nm to about 1000 nm in thickness. In certain embodiments, the metallic material coating may be a dusting of the metallic material. The coating may be uniform or non-uniform. The composition may further include magnesium chloride or silica at least partially applied over the metallic material coating. The composition may further include a sol-gel glass coating at least partially applied over the metallic material coating. The composition may, further include an adhesive to aid in adhesion of the metallic material to the bioactive glass ceramic material. The adhesive may be zirconium, titanium, chromium, or oxides thereof, other similar materials, and/or combinations thereof.

Certain other embodiments relate to a putty or paste that includes a composition described above mixed with water, saline, blood, or BMA.

Another embodiment relates to a bone grafting composite comprising a bioactive glass ceramic material, wherein the bone grafting composite is at least partially surface-coated with a metallic material. The metallic material is selected from the group consisting of gold, silver, platinum, copper, palladium, iridium, strontium, an isotope, an alloy or a combination thereof. The bioactive glass ceramic material is in a form of a particle, a sheet, a fiber, a strip, a block, a wedge, a mesh, or any combination of these forms. The metallic material is physically or chemically bound to the bioactive glass ceramic material.

Yet another embodiment relates to one or more bioactive glass sheets, strips, fibers, meshes, or composites surface-coated with a metallic material, such as gold, silver, platinum, copper, palladium, iridium, strontium, cerium, or isotopes, or alloys thereof. The metallic material may be physically (van der Waal forces, or hydrogen-bonding) or chemically (covalent bonds) bound to the bioactive glass sheets, strips, fibers, meshes or composites. In another embodiment, the surface coating comprises a process of vapor deposition of metallic material, such as gold, silver, platinum, copper, palladium, iridium, strontium, cerium, or isotopes, or alloys thereof onto at least a portion of the surface of the bioactive glass ceramic material. Alternatively, the surface coating comprises coating the bioactive glass ceramic material with a solution that includes the metallic material, such as gold, silver, platinum, copper, palladium, iridium, strontium, cerium, or isotopes, or alloys thereof and evaporating the solvent. In further alternative embodiment, the surface coating comprises sputter coating the bioactive glass ceramic with the metallic material, such as gold, silver, platinum, copper, palladium, iridium, strontium, cerium, or isotopes, or alloys thereof.

Yet another embodiment relates to a method for treating a wound comprising applying a synthetic bone grafting composition comprising a bioactive glass ceramic material at least partially surface coated with a metallic material, such as gold, silver, platinum, copper, palladium, iridium, strontium, cerium, or isotopes, or alloys thereof to the wound, wherein the wound comprises one or more of a bone injury and a soft tissue injury; and wherein the composition is effective to accelerate repair of the bone injury and the soft tissue injury. The composition is effective to reduce inflammation at the site of soft tissue injury. The composition is effective to control the coagulation of blood and/or other proteins at the site of the soft tissue injury. The composition is effective to conduct an electrical current to stimulate cellular activity and promote healing of the surrounding bone and soft tissues.

### DETAILED DESCRIPTION OF THE INVENTION

Inventors have discovered that metallic materials, such as gold, silver, platinum, copper, palladium, iridium, strontium, cerium, or isotopes, or alloys, or salts thereof, when surface-coated on at least a portion of a ceramic material, such as bioactive glass, are able to conduct an electrical current and prevent or reduce body's inflammatory response at or near the injury site upon the delivery of the metallic material-coated bioactive glass, enhancing the activity of the bioactive glass and the bone healing process. When bone is injured, it generates an electrical field. This low-level electrical field is part of the body's natural process that stimulates bone healing. When this healing process fails to occur naturally, a conductive implant material can facilitate regeneration of the bone. Conductive implants provide a safe, treatment that helps promote healing in fractured bones and spinal fusions which may have not healed or have difficulty healing. The devices stimulate the bone's natural healing process by sending low-level pulses of electromagnetic energy to the injury or fusion site.

As such, coating ceramic materials with a metallic material provides a solution to the problem of unwanted inflammatory response that may arise from an injury as well as the presence of bioactive glass. Also, by having a metallic material, such as gold coated on the surface of the ceramic material (rather than incorporated into the structure of the material), the surfaces becomes conductive and the gold becomes available to function in reducing inflammation immediately upon the delivery of the gold-coated ceramic material, e.g., bioactive glass particles to the delivery site within the body.

Metallic materials, such as gold are known to be highly conductive and possess anti-inflammatory properties. Importantly, electrical conductance and reduction of inflammation at the site of a wound may increase the rate at which the wound heals. Metallic materials may also promote wound healing by initiating or promoting angiogenesis. Increased blood flow may increase the rate of wound healing. Other benefits of gold may also be present.

The term “metallic material” refers to pure metals, such as gold, silver, platinum, copper, palladium, iridium, strontium, cerium, or isotopes (including radioisotopes), or alloys, or salts (the ionic chemical compounds of metals) thereof or other metallic materials having an atomic mass greater than about 45 and less than about 205. The term “atomic mass” is the mass of an atomic particle, sub-atomic particle, or molecule. It is commonly expressed in unified atomic mass units (u) where by international agreement, 1 unified atomic mass unit is defined as 1/12 of the mass of a single carbon-12 atom (at rest).

The term “metal alloy” refers to a material that's made up of at least two different chemical elements, one of which is a metal. The most important metallic component of an alloy (often representing 90 percent or more of the material) is called “the main metal,” “the parent metal,” or “the base metal.” The other components of an alloy (which are called “alloying agents”) can be either metals or nonmetals and they're present in much smaller quantities (sometimes less than 1 percent of the total). Although an alloy can sometimes be a compound (the elements it's made from are chemically bonded together), it's usually a solid solution (atoms of the elements are simply intermixed, like salt mixed with water). Examples of alloys include, e.g., bronze (copper (78-95%), tin (5-22%), plus manganese, phosphorus, aluminum, or silicon); amalgam (mercury (45-55%), plus silver, tin, copper, and zinc); steel (stainless; iron (50%+), chromium (10-30%), plus smaller amounts of carbon, nickel, manganese, molybdenum, and other metals), sterling silver (silver (92.5%), copper (7.5%)).

The term “metal isotopes” refers to variants of a particular chemical element which differ in neutron number, although all isotopes of a given element have the same number of protons in each atom. One example of a stable isotope of gold is gold-197 (¹⁹⁷Au). Examples of isotopes of copper include copper-63 (⁶³Cu) and copper-65 (⁶⁵Cu); examples of isotopes of iridium include iridium-192 (¹⁹²Ir) and iridium-193 (1⁹²Ir); examples of isotopes of palladium include palladium-102 (¹⁰²Pd), 104 (¹⁰⁴Pd), 105 (¹⁰⁵Pd), 106 (¹⁰⁶Pd), 108 (¹⁰⁸Pd) and 110 (¹¹⁰Pd); examples of isotopes of platinum include, e.g., five stable isotopes (¹⁹²Pt, ¹⁹⁴Pt, ¹⁹⁵Pt, ¹⁹⁶Pt, ¹⁹⁸Pt) and one very-long lived (half-life 6.50×10¹¹ years) radioisotope (¹⁹⁰Pt).; examples of isotopes of silver include two stable isotopes ¹⁰⁷Ag and ¹⁰⁹Ag with ¹⁰⁷Ag; examples of isotopes of strontium include four stable, naturally occurring isotopes: ⁸⁴Sr (0.56%), ⁸⁶Sr (9.86%), ⁸⁷Sr (7.0%) and ⁸⁸Sr (82.58%).

The term “metal salts” refers to the ionic chemical compounds of metals. For example gold salts include, e.g., sodium aurothiomalate and auranofin.

One aspect of the invention provides for a bone grafting composition comprising a bioactive glass ceramic material (that may be in the form of a particle, a sheet, a fiber, a mesh, or any combination of these forms) surface-coated with a metallic material. The composition may be synthetic. The terms “surface-coated” or “surface coating” refer to a covering (e.g., a film) that is applied to the surface of the bioactive glass ceramic material or a bioactive glass containing composite. A portion of the surface or substantially entire surface of the bioactive glass ceramic material or bioactive glass containing composite may be coated with a metallic material. The surface-coating may be uniform or non-uniform. The surface coating may be a dusting of coating. The surface coating may be a thin film (from 1 nm to 5000 nm in thickness) or a layer.

In certain embodiments, a thin film or layer of a metallic material, such as gold is coated on the bioactive glass ceramic material or a bioactive glass containing composite.

In certain embodiments, at least a portion of the bioactive glass ceramic material or bioactive glass containing composite is coated with a thin layer or film of metallic material such as gold; alternatively, substantially entire surface of the bioactive glass ceramic material may be coated with a thin layer or film of metallic material. For example, when the bioactive glass ceramic material is in a form of a particle, substantially entire surface of the particle is coated with a thin layer of gold. In another example, when the bioactive glass ceramic material is in a form of a porous block of material or a bioactive glass containing composite, substantially entire outer surface of the porous block of material is coated with a thin layer of gold.

In certain embodiments, the bioactive glass ceramic is coated with a thin layer of a film of metallic material such as gold without using an adhesion layer, such as chromium or titanium based adhesion layer.

In some embodiments, the metallic material and the bioactive glass together reduce the amount of inflammation in the bone and/or surrounding soft tissue. Bioresorbable implant conductivity and reduced inflammation may enhance the rate of both bone formation and soft tissue wound healing.

In the compositions described herein, the metallic material may be present in approximate amounts of 0.0001-20 wt. % ratio with reference to the total weight of the bioactive glass coated with the metallic material, or any amount in-between this ratio. Alternatively, the metallic material may be present in approximate amounts of, e.g., 0.000-10 wt. % ratio with reference to the total weight of the bioactive glass coated with the metallic material; 0.0001-5 wt % ratio with reference to the total weight of the bioactive glass coated with the metallic material; 0.0001-2.5 wt % ratio with reference to the total weight of the bioactive glass coated with the metallic material; or 0.0001-1 wt % ratio with reference to the total weight of the bioactive glass coated with the metallic material. The metallic material may also be present in a weight ratio of less than less than 20 wt %; less than 10 wt. %; less than about 5 wt. %; less than about 2.5 wt. %; less than about 1 wt. %; or less than about 0.5 wt. %. In some embodiments of this and other aspects of the invention, the weight ratio may be about 0.1%, about 0.2%, about 0.3%, about 0.4%, about 0.5%, about 0.6%, about 0.7%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, or about 5%. When making a putty, the metallic material may be present in an amount of less than about 2.5 wt % ratio or greater to ensure the putty is moldable and irrigation resistant. When preparing crosslinked sheets, sponges, and composites amounts of gold less than about 2.5 wt % may also be used.

Bioactive glass used in the invention may be melt-derived or sol-gel derived. Depending on their composition, bioactive glasses of the invention may bind to soft tissues, hard tissues, or both soft and hard tissues. The composition of the bioactive glass may be adjusted to modulate the degree of bioactivity. Furthermore, borate may be added to bioactive glass to control the rate of degradation. Additional elements, such as copper, zinc, silver and strontium may be added to bioactive glass to facilitate healthy bone growth. Bioactive glass that may also be suitable include glasses having about 40 to about 60 wt % SiO₂, about 10 to about 34 wt % Na₂O, up to about 20 wt % K₂O, up to about 5 wt % MgO, about 10 to about 35 wt % CaO, 0 to about 35 wt % SrO, up to about 20 wt % B₂O₃, and/or about 0.5 to about 12 wt % P₂O₅. The bioactive glass may additionally contain up to 10 wt % CaF₂.

Bioactive glass is capable of bonding to bone, which begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer is another layer which becomes increasingly silica rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

Application of bioactive glass to bone may promote bone remodeling. Bone remodeling occurs by equilibrium between osteoblast-mediated bone formation and osteoclast-mediated bone destruction. When bone is injured or missing, such as in a fracture, promotion of osteoblast activity is thought to be helpful to induce bone formation. Further, promoting bone formation by osteoblasts may be helpful in locations in which there is significant bone loss in the absence of an apparent injury.

The bioactive glass may have osteostimulative properties, which refers to promoting proliferation of the osteoblasts such that bone can regenerate. In an osteostimulative process, a bioactive glass material may be colonized by osteogenic stem cells. This may lead to quicker filling of bone defects than would otherwise occur with an osteoconductive glass.

In various embodiments of this and other aspects of the invention, the bioactive glass sheets, fibers, and mesh may provide structure to a tissue site in order to support, promote or facilitate new tissue growth.

The bonding of bioactive glass to bone begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer of the bioactive glass is another layer which becomes increasingly silica rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

Bioactive glass ceramic material may be in a form of particles, fibers, meshes, sheets, blocks or wedges. The bioactive glass ceramic material may be incorporated within a composite.

The bioactive glass particles can range in size from about 0.01 μm to about 5 mm.

Bioactive glass ceramic material may be porous or non-porous or a combination of porous and non-porous bioactive glass ceramic material.

The bioactive glass particles, fibers, meshes, sheets, blocks or wedges maybe prepared by a sol-gel method. Methods of preparing such bioactive active glasses are described in Pereira, M. et al., “Bioactive glass and hybrid scaffolds prepared by sol-gel method for bone tissue engineering” Advances in Applied Ceramics, 2005, 104(1): 35-42 and in Chen, Q. et al., “A new sol-gel process for producing Na₂O-containing bioactive glass ceramics,” Acta Biomaterialia, 2010, 6(10):4143-4153.

The composition can be allowed to solidify. In some embodiments, particles of bioactive glass are sintered to form a porous glass.

Repeated cooling and reheating may be performed on the solidified or sintered bioactive glass, with or without spinning, to draw the bioactive glass produced into fibers. A glass drawing apparatus may be coupled to the spinner and the source of molten bioactive glass, such as molten bioactive glass present in a crucible, for the formation of bioactive glass fibers. The individual fibers can then be joined to one another, such as by use of an adhesive, to form a mesh.

Alternatively, the bioactive glass in molten form may be placed in a cast or mold to form a sheet or another desired shape.

The bioactive glass ceramic material (glass particles, fibers, meshes, sheets, blocks or wedges) or a bioactive glass containing composite may further comprise any one or more of adhesives, grafted bone tissue, in vitro-generated bone tissue, collagen, calcium phosphate, stabilizers, antibiotics, antibacterial agents, antimicrobials, drugs, pigments, X-ray contrast media, fillers, and other materials that facilitate grafting of bioactive glass to bone.

A bioactive glass ceramic material suitable for the present compositions and methods may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass ceramic. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, and boron with only traces of the other(s). The term “about” as it relates to the amount of calcium phosphate present in the composition means +/−0.5%. Thus, about 5% means 5+/−0.5%.

The bioactive glass ceramic materials may further comprise one or more of a silicate, borosilicate, borate, strontium, or calcium, including SrO, CaO, P₂O₅, SiO₂, and B₂O₃. An exemplary bioactive glass is 45S5®, which includes 46.1 mol % SiO₂, 26.9 mol % CaO, 24.4 mol % Na₂O and 2.5 mol % P₂O₅. An exemplary borate bioactive glass is 45S5B1, in which the SiO₂ of 45S5 bioactive glass is replaced by B₂O₃. Other exemplary bioactive glasses include 58S, which includes 60 mol % SiO₂, 36 mol % CaO and 4 mol % P₂O₅, and S70C30, which includes 70 mol % SiO₂ and 30 mol % CaO. In any of these or other bioactive glass materials described herein, SrO may be substituted for CaO.

The following composition, having a weight % of each element in oxide form in the range indicated, will provide one of several bioactive glass compositions that may be used to form a bioactive glass ceramic:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

The bioactive glass ceramic materials can be in the form of a three-dimensional compressible body of loose glass-based fibers in which the fibers comprise one or more glass-formers selected from the group consisting of P₂O₅, SiO₂, and B₂O₃. Some of the fibers have a diameter between about 100 nm and about 10,000 nm, and a length:width aspect ratio of at least about 10. The pH of the bioactive glass can be adjusted as-needed.

In some embodiments, the body comprises fibers having a diameter between about 100 nm and about 10,000 nm. The especially small diameter of these fibers renders them highly flexible so they form into the compressible body without breaking. In some embodiments the body includes fibers meeting these dimensional requirements in addition to other glass morphologies, such as fibers of other dimensions, microspheres, particles, ribbons, flakes or the like. The fibers may have a variety of cross section shapes, such as flat, circular, oval, or non-circular.

Bioactive glass ceramics may be prepared by heating a composition comprising one or more of SiO₂, CaH(PO₄), CaO, P₂O, Na₂O, CaCO₃, Na₂CO₃, K₂CO₃, MgO, and H₂BO₃ to a temperature between 1300 and 1500° C. such that the composition may form molten glass. An exemplary composition that can form fibers includes 40-60% SiO₂, 10-20% CaO, 0-4% P₂O₅, and 19-30% NaO. Other exemplary compositions include 45S5, which includes 46.1 mol % SiO₂, 26.9 mol % Ca), 24.4 mol % Na₂O and 2.5 mol % P₂O₅; 45S5B1, which includes 46.1 mol % B₂O₃, 26.9 mol % Ca), 24.4 mol % Na₂O and 2.5 mol % P₂O₅; 58S, which includes 60 mol % SiO₂, 36 mol % CaO and 4 mol % P₂O₅; and S70C30, which includes 70 mol % SiO₂ and 30 mol % CaO. Another exemplary composition includes 40 mol % SiO₂, 40 mol % B₂O₃, 20 mol % CaO, and 20 mol % Na₂O.

In some embodiments, the bioactive glass ceramic material (e.g., a particle, sheet, fiber, or mesh) is treated with certain buffer solutions to prepare the surface of the bioactive glass material for application of gold, cell adhesion and/or control pH prior to the exposure of the particles with cells.

In some embodiments, the bioactive glass ceramic material is treated with the buffer solution or solutions before the metallic coating is applied to the material. In this context, the bioactivity and bone formation using the glass, fibers, mesh, or ceramic may be enhanced by treating these with a buffer solution. The glass, fibers, mesh, or ceramic may be buffer-treated and dried before addition of the metallic coating.

In certain embodiments, the pre-treatment buffer solution has a starting pH of from about 6 to about 12 but may reach an end pH of about 9.5. Examples of buffers that might be suitable for the pre-treatment of the present invention include mixed sodium phosphate salts (such as Sorensen's Phosphate buffer, Millonig's Phosphate buffer, Karlsson and Shultz Phosphate buffer, Maunsbach Phosphate buffer, and Phosphate Buffered Saline (PBS); buffer pH of about 6.4-8.0), TAPS (3-{[tris(hydroxymethyl)methyl]amino}propanesulfonic acid; buffer pH of about 7.7-9.1), Bicine (N,N-bis(2-hydroxyethyl)glycine; buffer pH of 7.6-9.0), Tricine (N-tris(hydroxymethyl)methylglycine; buffer pH about 7.4-8.8), Tris (tris(hydroxymethyl)methylamine; buffer pH of about 7.5-9.0), HEPES (4-2-hydroxyethyl-1-piperazineethanesulfonic acid; buffer pH of about 6.8-8.2), TES (2-{[tris(hydroxymethyl)methyl]amino}ethanesulfonic acid; buffer pH of about 6.8-8.2), MOPS (3-(N-morpholino)propanesulfonic acid; buffer pH of about 6.5-7.9), PIPES (piperazine-N,N′-bis(2-ethanesulfonic acid); buffer pH of about 6.1-7.5), Cacodylate (dimethylarsinic acid; buffer pH of about 5.0-7.5), SSC (saline sodium citrate; buffer pH of about 6.5-7.5), or MES (2-(N-morpholino)ethanesulfonic acid; buffer pH of about 5.5-6.7). Any other buffer having appropriate pH buffering range of about 6 to about 12 might be suitable.

In certain embodiments, the end pH does not exceed 9.5, 9.4, 9.3, 9.2, 9.1, 9.0, 8.8, 8.9, 8.7, 8.6, 8.5, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, or 6.0. The end pH may range from 6.0 to 9.5.

Depending on the buffer used, the bioactive glass materials may be pretreated for different periods such that they become suitable for bone regeneration. Pre-treating the bioactive glass materials much longer than necessary to activate them may lead to deactivation. Similarly, if the bioactive glass materials are not pre-treated long enough, they may remain too active. In some embodiments, bioactive glass materials may be pretreated with the buffer for as short as 30 minutes. Other embodiments of the bioactive glass materials may require pretreatment as long as 24 hours. In some embodiments, the bioactive glass materials may be pretreated about 1 to about 2 hours, about 3 to about 4 hours, about 5 to about 6 hours, about 7 to about 8 hours, about 9 to about 10 hours, about 11 to about 12 hours, about 13 to about 14 hours, about 15 to about 16 hours, about 17 to about 18 hours, about 19 to about 20 hours, about 21 to about 22 hours, or about 23 to about 24 hours. Some bioactive glass materials may require pretreatments longer than 24 hours. As used here in the context of pre-treatment time, the term “about” means +/−30 minutes. A person skilled in the art can easily design simple experimental procedures to determine the optimum pretreatment time for any given bioactive glass material that is to be coated with a gold. After pretreatment, the bioactive glass material may be dried before being coated with gold.

In another embodiment of this aspect, silicate ions are released into and/or onto the wound and/or bone defect. In yet another embodiment of this aspect, calcium ions are released into the bone. In a further embodiment, borate ions are released into the bone. In any of the above embodiments of this aspect of the invention, sufficient ions, which include but are not limited to silicate, calcium, and borate, are released from the bioactive glass ceramic into the bone defect to achieve a critical concentration of ions to stimulate the proliferation and differentiation of an osteoblast and/or are released into the tissue at the site of the wound to promote wound healing.

In certain further embodiments, the bioactive glass ceramic material (e.g., a particle, sheet, block, wedge, fiber, or mesh) may include an adhesive layer to aid in coating the ceramic material with a metallic material. Exemplary materials that may be used for coating the bioactive glass ceramic material to improve adhesion of gold to the material include zirconium, titanium, chromium, or oxides thereof, and/or combinations thereof, and other suitable adhesive materials known to a skilled artisan.

In certain further embodiments, depending on the intended use of the compositions, the metallic material-coated bioactive glass ceramic material may include additional coating layers applied after the metallic material layer is applied and over the metallic material layer. For example, for dental applications, the gold-coated bioactive glass ceramic material may be coated with magnesium fluoride. For example, for bone injury applications, the gold-coated bioactive glass ceramic material may be coated with silica.

In certain embodiments, the bioactive glass ceramic materials (particles, fibers, blocks, wedges, sheet or mesh) can further comprise a carrier or a graft extender. The carrier may be one or more of hydroxyapatite, tricalcium phosphate, and calcium salts such as, but not limited to, calcium sulfate and calcium silicate. The bioactive glass may be in a granular form and comprise other materials as carriers as well.

In some embodiments, the bioactive glass fibers forming the bioactive glass sheet may be arranged in a variety of structures to form a wrap. In these structures, the bioactive glass fibers may be woven, knitted, warped knitted, and/or braided. The bioactive glass fibers can also form a mesh-like structure. The bioactive glass sheet may be formed such that it has a substantially greater length than width.

In any of the woven, knitted, warped knitted, or braided patterns, the bioactive glass fibers are in both a longitudinal and transverse orientation. For example, the longitudinal fibers may be interwoven with transverse fibers. Some transverse fibers can be wrapped around the outside of the longitudinal fibers to secure the longitudinal and transverse fibers. In one example, the transverse fibers may be wrapped around the longitudinal fibers to form a knot or whipping. Alternatively, the longitudinal fibers may be stitched to the transverse fibers.

The openings within the sheet or mesh may have a low density. The structure and density of the bioactive glass fibers may be similar to the density of material in VICRYL (Ethicon, Inc., Somerville, N.J.). The sheet or mesh may have any one or more of about 5%, 10%, 15%, 20%, 25%, 30%, 35%, 40%, 45%, 50%, 55%, 60%, 65%, 70%, 75%, 80%, 85%, 90%, and 95% empty opening as a percentage of the area. The density of the mesh or sheet may be sufficiently high, i.e. the openings must have a low enough percentage area, such that the wrap is able to be sutured. The density may also be high enough such that the wrap serves as a barrier to hyperplasis and tissue adhesion.

The bioactive glass ceramic materials (particles, fibers, blocks, wedges, sheets or mesh) may have binding regions, which may enable the materials to be anchored to the bone. For example, a bone anchoring device can affix or anchor the sheet or mesh by extending through the sheet or mesh. Exemplary bone anchoring devices include screws, sutures, stables, pins, buttons, and combinations thereof. The bone anchoring device can be attached to a drilled or hollowed out region of bone.

The bone defect may be a fracture or may result from an injury or bone disease, such as osteoporosis.

The bioactive glass may release one or more of silicate, calcium, and borate ions into the bone defect. Ions released into the bone defect can stimulate osteoblast activity.

In certain embodiments, the bioactive glass ceramic material may further comprise a carrier, such as hydroxyapatite and tricalcium phosphate, or a graft extender.

The bioactive glass ceramic provided by this aspect of the invention may be effective to produce hydroxyapatite in a hard tissue and to promote wound healing. An exchange of ions may occur between bioactive glass and the surrounding body fluid that results in the production of hydroxyapatite. The exchanged ions may also enhance the rate of wound healing, such as by stimulating collagen production and/or by activating genes responsible for cell proliferation at the site of a wound. The ions exchanged may be any one or more of silicate, calcium, and borate.

Gold is knows to possess anti-inflammatory properties. Thus, incorporation of gold onto the surface of bioactive glass ceramics can serve to prevent or minimize body's immune response, particularly in the context of wounded tissue at or near the site of a bone injury. Further, gold on the surface of bioactive glass ceramics may even be helpful to heal wounds not associated with a bone injury.

Local sources of calcium, magnesium, zinc, silica, borate, strontium, silver and other ions that may be included in and/or released by bioactive glass may enhance the rate of wound healing. The presence of additional calcium, magnesium, silica, sodium, borate and zinc ions, in particular, may serve to signal cells to enhance the rate of wound healing. Silica ions, along with the increased pH arising from release of sodium ions are conducive to wound healing. In addition, borate ions may promote wound healing. Silver ions may be effective to reduce inflammation and to inhibit bacterial growth. With regard to bone repair, the presence of calcium and silica ions at critical concentrations near the bone can activate genes responsible for osteo progenitor cells to differentiate into osteoblasts. See, e.g., Jones, J. R. et al, “Extracellular matrix formation and mineralization on a phosphate-free porous bioactive glass scaffold using primary human osteoblast (HOB) cells” Biomaterials, 2007, 28(9): 1653-63.

In some embodiments, pure metals, metal alloys, metal isotopes or radioisotopes, or salts formed therefrom may be bound to the bioactive glass. The metallic material may be physically (van der Waal forces, or hydrogen-bonding) or chemically (covalent bonds) bound to the bioactive glass ceramic material. Such bonding may occur by any means known to one skilled in the art, including but not limited to, the formation of covalent bonds, van der Waal forces, or hydrogen-bonding. Gold is utilized in the following specific examples to further illustrate the bone grafting compositions and should not be construed to limit the scope of the disclosure. The metals may include other precious metals without departing from or exceeding the spirit or scope of the disclosure. The surface of gold, gold alloys, and gold isotopes or radioisotopes may be functionalized with complexes or compounds that have carboxylic acid groups, hydroxyl groups, thiol groups, phosphate groups, or amide functional groups, to name a few, that can be used to form covalent bonds with bioactive glass through the use of a coupling agent. An exemplary coupling agent is aminopropyl silane. Such coupling agents are available from Gelest Inc., for example. Other coupling agents include amine, sulfur, phosphorus, epoxy, hydride and carboxylate agents. Specific examples of coupling agents include, but are not limited to, aminopropyl triethoxysilane, diaminopropyl diethoxysilane, glycidoxypropyl trimethoxysilane, aminopropyl trimethoxysilane, aminopropyl triethoxysilane, carboxyethylsilanetriol, triethoxysilylpropylmaleamic acid, N-(trimethoxysilylpropyl)ethylene diamine triacetic acid, 3-(trihydroxysilyl)-1-propane sulfonic acid, and 2-(4-chlorosulfonylphenyl)ethyltrimethoxysilane. Additional coupling agents include amine, sulfur, phosphorus, epoxy, hydride and carboxylate agents. When these coupling agents are used, the trialkoxy groups may directly react with the surface of the glass or hydrolyze to form hydroxyl groups that react with the surface of the glass through the formation of hydrogen bonds or covalent linkages, while the amino portion of the coupling agent interacts with the gold, gold alloys, salts or radioisotopes. The end result is the bonding of the gold, gold alloys, salts or radioisotopes to the bioactive glass.

As gold is a metal, in certain embodiments, it can form an alloy with other metals. For example, gold may form an alloy with silver, copper, rhodium, nickel, platinum, palladium, zinc, or aluminum, to name a few.

In various embodiments, the metallic materials, metallic material alloys, salts or radioisotopes need not remain bound to the bioactive glass after implantation of a metallic material-coated bioactive glass ceramic material into the body. In the body, the gold may eventually be disassociated from the bioactive glass. The bioactive glass and the metallic material would both be present in the tissue near the implant site. Both substances can then promote healing of the wound at the implant site. The advantage of the metallic material such as gold being coated on the surface of the bioactive glass ceramic is that the gold becomes available immediately upon implantation to the body (rather than as the glass dissolves) to help with any anti-inflammatory response at the site of the implantation as well as around the site. Without being bound by any particular mechanism, the bioactive glass may promote bone repair and induce soft tissue repair by the release of calcium ions. The metallic material, e.g., gold, may promote immediately aid in reducing inflammation, and/or counteract any tendency of the bioactive glass in the wound site to promote coagulation, promote angiogenesis, and enhance soft tissue repair.

In any of the embodiments of this aspect, the composition including metallic material-coated bioactive glass ceramic material promotes more rapid wound healing than that achieved by an uncoated non-conductive bioactive glass ceramic material. The metallic material, such as gold present on the bioactive glass serves to conduct electrical current, reduce the inflammation and enhance the rate of wound healing. Further, conductivity of the implant material along with the ions released by the bioactive glass combined with the activity of the gold may synergistically enhance the rate of wound healing. Synergy may arise from any one or more of the following metallic material activities: anti-inflammatory activity, reduction of blood clotting and/or coagulation, facilitation of the migration of cells into the scaffold, formation of blood vessels, and stimulation of genes to increase the rate of healing of hard and soft tissues.

Another aspect relates to a material comprising a natural or synthetic polymer and bioactive glass, with the bioactive glass or the resultant composite coated with a thin film or layer of metallic material, such as gold. The metallic material may be physically or chemically bound to the bioactive glass. The bioactive glass may be in the form of a particle, a glass sheet, a fiber, block, wedge, strip, a mesh, or any combination of these forms. The polymer may include but are not limited to the copolymers (PLGA) of poly(lactic acid) (PLA) and poly(glycolic acid) (PGA), which have been widely used synthetic polymeric materials, because of their controllable degradation rate and mechanical properties. Other polymers for bone tissue engineering include, but are not limited to, polyanhydrides, polycarbonates, polyphosphazenes, polycaprolactone and polyfumarates, polyesters, polyurethanes, polyalkyleneoxides, polyethers polyamides, copolymers and combinations thereof. Examples of natural polymers may include and are not limited to collagen, gelatin, polysaccharides, alginates, polypeptides, polyaminoacids and combinations thereof. Naturally produced ceramics, such as corals may also be used for the repair of bones. Corals have good biocompatibility, well-interconnected porous structure, and appropriate mechanical properties. Synthetic calcium-based ceramics such as hydroxyapatite (HA) and hydroxyapatite-tricalciumphosphate are also osteoconductive materials and can also be used. When the bioactive calcium-based ceramics are combined with polymers, scaffold mechanical properties as well as the osteoconductivity can be improved as demonstrated by many composite-based scaffolds such as collagen-HA-PLGA, chitosan-hydroxyapatite, PLA-polyethyleneglycol (PEG), collagen-PLA-HA, and polycaprolactone (PCL)-HA.

In further embodiments, the composition including metallic material-coated bioactive glass ceramic material may be a composite material. The composite material may include a natural or synthetic polymer and bioactive glass, with the bioactive glass or resultant composite coated with a thin layer or film of a metallic material. The composite material may also include glycosaminoglycans. The glycosaminoglycans may be coated and/or ionically or covalently bound to the bioactive glass ceramic material and may be any one or more of heparin, heparin sulfate, chondroitin sulfate, dermatan sulfate, keratan sulfate, and hyaluronic acid. These glycosaminoglycans may further enhance the rate of wound healing and/or bone formation. The enhancement of wound healing and/or bone formation may be synergistic.

In some embodiments, the coated bioactive glass compositions bioresorb at a rate approximately equivalent to the rate of formation of new bone at or near the site of the bone defect. Such bioresorption may result from a significant contribution of ions from the bioactive glass to the surrounding tissue and/or bone such that the ions are incorporated into the tissue and/or bone. It may also be the case in some embodiments that the rate of mass increase of the bone at or near the site of the bone defect is consistent with the rate at which the mass of the composition decreases. The rate of bioresorption may be controlled by altering the size of the bioactive glass particles and/or the composition of ions within the bioactive glass particles along with selection and degree of crosslinking in bioactive glass containing composites.

Another embodiment relates to a method for treating a wound. Bioactive glass ceramic material coated with a metallic material, such as, e.g., gold, is applied to the wound. The bioactive glass ceramic material may be in the form of a particle, a glass sheet, a fiber, a mesh, block, wedge, strip, or other shape or a bioactive glass containing composite of varying shape or size. The preparation of the particle, sheet, fiber, mesh, block, wedge, strip or other shape may be undertaken as described above. The wound comprises one or more of a bone injury and a soft tissue injury. The coated bioactive glass ceramic material is effective to accelerate repair of the bone injury and the soft tissue injury.

Another embodiment provides for a method of treating a bone defect. A bioactive glass ceramic material coated with a metallic material is applied to the site at or near the bone defect. The bioactive glass may be in the form of a particle, a glass sheet, a fiber, a block, a wedge, a strip, a mesh, or any combination of these forms. The coated bioactive glass ceramic material is bioresorbable at a rate consistent with the rate of formation of new bone at or near the site.

Another embodiment provides for a method of stimulating the activity of a gene that promotes wound healing and/or bone regeneration. Bioactive glass ceramic material coated with a metallic material is applied to the site at or near the bone defect. The bioactive glass may be in the form of a particle, a glass sheet, a block, a wedge, a strip, a fiber, a mesh, or any combination of these forms or a bioactive glass containing composite of varying shape or size. The activity of the gene is stimulated. The gene may be one or more of BMP-2, Runx2, Osterix, Dlx5, TGF-beta, PDGF, VEGF, collagen I, ALP (alkaline phosphatase), bone sialoprotein, PINP (procollagen type I N-terminal propeptide), osteoponin, osteonectin, and osteocalcin.

BMP-2, also known as bone morphogenetic protein 2, is a member of the TGF-beta superfamily of proteins. Stimulation of BMP-2 activity, such as by stimulating the BMP-2 gene and/or protein expression, can lead to stimulation of bone production. BMP-2 stimulation may enhance the overall rate and extent of bone defect repair.

Runx2, also known as Runt-related transcription factor 2, is a transcription factor that is associated with osteoblast development and differentiation. Mutations in the Runx2 gene are associated with Cleidocranial dysostosis, a general skeletal condition. Stimulation of Runx2 activity, such as by stimulating the Runx2 gene and/or expression of its associated protein, can lead to stimulation of bone production. Runx2 stimulation may enhance osteoblast formation and activity, as well as the overall rate and extent of bone defect repair.

Osterix is a transcription factor that plays a role in osteoblast differentiation and bone formation. As discussed in Cao et al., Cancer Res., 2005, 65:1124-8, Osterix may play a role in osteoblast differentiation and tumor activity in osteosarcoma. Stimulation of Osterix activity, such as by stimulating the Osterix gene and/or protein expression, can lead to stimulation of bone production. Osterix stimulation may enhance the overall rate and extent of bone defect repair.

DLX-5 is a protein that is encoded by the homeobox transcription factor gene DLX5. Mutations in DLX-5 may be associated with hand and foot malformations. Stimulation of DLX-5 protein expression and/or activity, as well as stimulation of DLX5 gene expression, may lead to stimulation of bone production and enhancement of bone defect repair.

TGF-beta (transforming growth factor beta) is a protein that exists in three isoforms, TGF-beta1, TGF-beta2, and TGF-beta3. Genes encoding these proteins include TGFB1, TGFB2, and TGFB3. Activation of these genes, as well as enhancement of the activity of the TGF-beta proteins, can promote tissue remodeling. Increased tissue remodeling can serve to enhance the rate of tissue repair and wound healing.

PDGF (platelet-derived growth factor) is a growth factor that regulates cell growth and division. PDGF plays a major role in angiogenesis, as well as cell proliferation, cell migration, and embryonic development. Gold may serve to increase PDGF activity as a means to promote wound healing by any one or more of these mechanisms. PDGF is found as four ligands, PDGFA, PDGFB, PDGFC, and PDGFD. These ligands may form dimers. Also, PDGFA and PDGFB may form a heterodimer.

VEGF (vascular endothelial growth factor) is a family of growth factors that include VEGF-A, VEGF-B, VEGF-C, VEGF-D, and PGF (placenta growth factor). VEGF stimulates angiogenesis and promotes cell migration, both processes useful in the repair of soft-tissue wounds. A metallic material may promote VEGF-mediated activity. Further, in various embodiments, drugs such as bevacizumab and ranibizumab, which enhance VEGF activity, may be included in the GAG-bioactive glass compositions.

Collagen I, also known as type-I collagen, is found both in scar tissue and in the organic part of bone. Collagen I is also found in tendons and the endocmysium of myofibrils. Stimulation of collagen I production, such as by stimulating expression of genes associated with collagen I, including COLIA1 and COLIA2, may enhance the overall rate and extent of bone defect repair.

ALP, also known as ALKP and alkaline phosphatase, removes phosphate groups from many types of molecules. ALPL, an alkaline phosphatase isozyme, is found in various tissues of the human body, including bone. Stimulation of ALP and/or ALPL activity, may lead to stimulation of bone production. ALP and/or ALPL stimulation may enhance the overall rate and extent of bone defect repair.

Bone sialoprotein, also known as BSP, cell-binding sialoprotein or integrin-binding sialoprotein, is a significant component of bone extracellular matrix. The IBSP gene encodes bone sialoprotein. Stimulation of IBSP gene expression and/or bone sialoprotein expression, may enhance the overall rate and extent of bone defect repair. For example, bone sialoprotein could improve the mineralization of newly-formed bone matrix at the repair site.

Procallagen type I N-terminal propeptide, also known as PINP, is an effective marker of bone formation as this gene promotes collagen turnover. PINP expression is proportional to the amount of new collagen laid down when bone is formed. Stimulation of PINP gene expression and/or PINP protein expression may enhance the overall rate and extent of bone defect repair by enhancing the rate of collagen deposition in the bone.

Osteopontin, also known as BSP-1, ETA-1, SPP1, 2ar, and Ric, is a protein expressed in bone, as well as other tissues. Ostepontin is synthesized by fibroblasts, preosteoblasts, osteoblasts, osteocytes, bone marrow cells, and endothelial cells. Osteopontin is known to be important in bone remodeling, such as by anchoring osteoclasts to the bone mineral matrix. Stimulation of osteopontin gene expression and/or osteopontin protein expression, may enhance the overall rate and extent of bone defect repair by enhancing the rate of bone formation.

Osteonectin, also known as SPARC or BM-40, is a protein encoded by the SPARC gene. Osteonectin binds sodium and is secreted by osteoblasts during bone formation. Osteonectin is thought to play an important role in bone mineralization and collagen binding. As high levels of osteonectin are detected in active osteoblasts, stimulation of SPARC gene expression and/or osteonectin protein expression may enhance the overall rate and extent of bone defect repair by enhancing the rate of bone formation.

Osteocalcin, also known as BGLAP, is a bone protein encoded by the BGLAP gene. Osteocalcin is secreted by osteoblasts and may play a role in bone mineralization. Stimulation of osteocalcin protein expression and/or BGLAP gene expression may enhance the overall rate and extent of bone defect repair.

Another aspect provides for a method of reducing inflammation at or near the site of a wound and/or a bone defect. Bioactive glass surface-coated with a metallic material is applied to the site of the wound or at or near the bone defect. Upon the application, the metallic material becomes immediately available and effective in reducing inflammation and pain and/or discomfort at or near the site of the wound and/or bone defect.

In some aspects, a compound bone fracture may be treated with the compositions described herein. A bone at the site of the compound bone fracture is wrapped with any of the above-described compositions of bioactive glass ceramic material surface-coated with a metallic material. The bioactive glass ceramic material may be in the form of fibers, a fiber mesh, and a sheet. The compositions may have enhanced anti-inflammatory activities that serve to reduce pain and discomfort in the surrounding wounded tissue as the compound bone fracture heals.

The metallic material-coated bioactive glass fibers, meshes, and sheets may be wrapped completely around the bone such that the ceramic is secured to the bone and/or maintains the bone shape so as to prevent further fracturing. One exemplary form of the bioactive glass ceramic is in the form of a mesh that can be wrapped around a large portion of bone surrounding the compound fracture so as to both provide pressure to the bone and to allow for the migration of ions from the mesh wrap into the bone. The bioactive glass ceramic may also be secured to the bone by one or more plates and/or one or more screws.

Another embodiment provides for a method of preparing a composition comprising bioactive glass surface-coated with a metallic material. A metallic material can be coated onto at least a portion of the surface of the bioactive glass ceramic material by methods known in the art.

For example, one method includes coating the bioactive glass ceramic material by means of dipping or spraying the bioactive glass ceramic material with a solution containing a metallic material. For example, the solution can be spray applied or poured onto/over the bioactive glass ceramic material (glass particles, fibers, sheets, etc.). Porous or non-porous blocks of bioactive glass can be dipped into a solution of metallic material. The glass can then be dried using a variety of techniques, including but not limited to freeze drying, vacuum drying, oven drying, and spray drying. The process can be repeated until the desired ratio of metallic material to glass is achieved.

Another method of coating metallic material onto the bioactive glass materials includes sputter deposition, which is a physical vapor deposition (PVD) method of thin film deposition by sputtering. This involves ejecting material from a “target” that is a source onto a “substrate” such as a bioactive glass ceramic material. PVD includes a variety of vacuum deposition methods that can be used to deposit thin films of metallic material by the condensation of a vaporized form of metallic film material onto various bioactive glass ceramic materials. The coating method involves purely physical processes such as high-temperature vacuum evaporation with subsequent condensation, or plasma sputter bombardment rather than involving a chemical reaction at the surface to be coated as in chemical vapor deposition.

Another method includes a sputter deposition process to cover the bioactive glass ceramic material with a thin layer of metallic material, such as, e.g., such as gold or a gold/palladium (Au/Pd) alloy.

In various embodiments, the metallic material need not remain bound to the bioactive glass ceramic material after implantation of a gold-coated bioactive glass ceramic material into the body. Preferably, in the body, the metallic material coating becomes immediately available for reducing inflammation at the implantation site. The bioactive glass and metallic material would both be present in the tissue near the implantation site. Both substances can then promote healing of the wound at the implant site. Without being bound by any particular mechanism, the bioactive glass may promote bone repair and induce soft tissue repair by the release of calcium ions. The metallic material may inhibit or reduce the inflammation, promote angiogenesis, enhance soft tissue repair, and/or counteract any tendency of the bioactive glass in the wound site to promote coagulation.

In some embodiments, the bioactive glass compositions may contain radioactive materials. Such bioactive glass compositions may be useful to treat tumors and bone defects arising out of cancer. The radiation emitted from the bioactive glass composition is effective to kill cancer cells within the tumors and bone defects.

Compositions in accordance with the disclosure may also be sterilized by, for example, aseptic processing and ethylene oxide sterilization.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents that are intended to define the spirit and scope of this invention.

1. A bone grafting composition comprising a bioactive glass ceramic material at least partially surface-coated with a metallic material having an atomic mass greater than 45 and less than 205.

2. The bone grafting composition of claim 1, wherein the metallic material is selected from the group consisting of gold, silver, platinum, copper, palladium, iridium, strontium, cerium, an isotope, an alloy or a combination thereof.

3. The bone grafting composition of claim 1, wherein the bioactive glass ceramic material is in a form of a particle, a sheet, a fiber, a strip, a block, a wedge, a mesh, or any combination of these forms.

4. The composition of claim 1, wherein the metallic material is physically or chemically bound to the bioactive glass ceramic material.

5. The composition of claim 1, wherein the bioactive glass ceramic material comprises at least one of silica, boron, and calcium phosphate.

6. The composition of claim 1, wherein the weight ratio of the metallic material is less than about 20% relative to the weight of the synthetic bone grafting composition.

7. The composition of claim 1, wherein the weight ratio of the metallic material is about 0.0001-20 wt % relative to the weight of the synthetic bone grafting composition.

8. The composition of claim 1, wherein the weight ratio of the metallic material is 0.0001-10 wt % relative to the weight of the synthetic bone grafting composition.

9. The composition of claim 1, wherein the weight ratio of the metallic material is 0.0001-5 wt % relative to the weight of the synthetic bone grafting composition.

10. The composition of claim 1, wherein the weight ratio of the metallic material is 0.0001-2.5 wt % relative to the weight of the synthetic bone grafting composition.

11. The composition of claim 1, wherein the weight ratio of the metallic material is 0.0001-1 wt % relative to the weight of the synthetic bone grafting composition.

12. The composition of claim 1, wherein the composition comprises 0-90% silica, 0-90% boric acid, or a combination thereof.

13. The composition of claim 1, wherein the bioactive glass ceramic material comprises bioactive glass.

14. The composition of claim 13, wherein the bioactive glass is in a form of particles ranging in size from about 0.1 nm to about 5 mm.

15. The composition of claim 13, wherein the bioactive glass comprises SiO₂, or P₂O₅, or B₂O₃, or SiO₂ and P₂O₅, or SiO₂ and B₂O₃, or K₂O, or MgO.

16. The composition of claim 13, wherein the bioactive glass comprises 40-60% SiO₂, 10-20% CaO, 0-4% P₂O₅, and 19-30% NaO.

17. The composition of claim 13, wherein the bioactive glass further comprises a carrier selected from the group consisting of hydroxyapatite, tricalcium phosphate, calcium sulfate, calcium silicate or calcium carbonate.

18. The composition of claim 1, wherein the bioactive glass ceramic material is pretreated in a solution comprising one or more of blood, bone marrow aspirate, bone-morphogenetic proteins, platelet-rich plasma, and osteogenic proteins.

19. The composition of claim 1, wherein the composition is in a form of a gel, putty, or a solid at a room temperature.

20. The composition of claim 1, wherein the composition is osteoinductive.

21. The composition of claim 1, wherein the composition conducts an electrical current.

22. The composition of claim 1, wherein the composition promotes more rapid wound healing as compared to a composition having uncoated bioactive glass ceramic material.

23. The composition of claim 1, wherein the metallic coating mount ranges from about 1 nm to about 5000 nm in thickness.

24. The composition of claim 1, further comprising magnesium chloride or silica at least partially applied over the metallic material coating.

25. The composition of claim 1, further comprising a sol-gel glass coating at least partially applied over the metallic material coating.

26. The composition of claim 1, further comprising an adhesive to aid in adhesion of the metallic material to the bioactive glass ceramic material.

27. The composition of claim 1, wherein the adhesive is selected from the group consisting of zirconium, titanium, chromium, oxides thereof, and combinations thereof.

28. The composition of claim 1, wherein the surface coating comprises a process of vapor deposition of metallic material onto at least a portion of the surface of the bioactive glass ceramic material.

29. The composition of claim 1, wherein the surface coating comprises coating the bioactive glass ceramic material with a solution comprising the metallic material.

30. A method for treating a wound comprising applying the composition of claim 1 to the wound, wherein the wound comprises one or more of a bone injury and a soft tissue injury; and wherein the composition is effective to accelerate repair of the bone injury and the soft tissue injury.

31. The method of claim 30, wherein the composition is effective in reducing inflammation at the site of soft tissue injury.

32. The method of claim 30, wherein the composition is effective in controlling coagulation of blood and/or other cells at the site of the soft tissue injury.

33. A putty or a paste comprising the composition of claim 1 mixed with water, saline, blood, or BMA.

34. A bone grafting composite comprising a bioactive glass ceramic material, wherein the bone grafting composite is at least partially surface-coated with a metallic material.

35. The bone grafting composite of claim 34, wherein the metallic material is selected from the group consisting of gold, silver, platinum, copper, palladium, iridium, strontium, cerium, an isotope, an alloy or a combination thereof.

36. The bone grafting composite of claim 34, wherein the bioactive glass ceramic material is in a form of a particle, a sheet, a fiber, a strip, a block, a wedge, a mesh, or any combination of these forms.

37. The bone grafting composite of claim 34, wherein the metallic material is physically or chemically bound to the bioactive glass ceramic material.

38. The bone grafting composite of claim 34, wherein the composite is osteoinductive.

39. The bone grafting composite of claim 34, wherein the metallic material conducts an electrical current.

40. The bone grafting composite of claim 34, wherein the composite promotes more rapid wound healing as compared to an uncoated bone grafting composite.

41. The composite of claim 34, wherein the metallic coating mount ranges from about 1 nm to about 5000 nm in thickness.
